(19) United States
(12) Patent Application Publication
(10) Pub. No.: US 2008/0009718 A1
(43) Pub. Date: Jan. 10, 2008

(54) Implements and methods for applying radiopaque markings

(76) Inventor: Gary Louis Zohman, Calabasas, CA (US)

Correspondence Address: LAW OFFICE OF ALAN W. CANNON, 942 MESA OAK COURT, SUNNYVALE, CA, 94086, US

(21) Appl. No.: 11/433,327

(22) Filed: May 11, 2006

(51) Int. Cl. A61B 5/05

(52) U.S. Cl. 600/426

(57) ABSTRACT

A marking implement for making a radiopaque marking to facilitate a surgical procedure. A main body is formed of a composition adapted to be smeared onto a substrate when dragged along the substrate. The composition including a radiopaque component that is applied to the substrate in the smear. A method of facilitating a surgical procedure includes subjecting a target surgical site within a patient to x-ray exposure for visualization of the target surgical site, locating a position on the skin of the patient relative to the target surgical site; and marking the position on the skin using a marking implement having a main body formed of a composition adapted to be smeared onto the substrate when dragged along the substrate and which includes a radiopaque component. A method of facilitating a surgical procedure includes locating a position on the skin of a patient relative to a target surgical site within the patient, marking the position on the skin using a marking implement having a main body formed of a composition adapted to be smeared onto the substrate when dragged along the substrate and which includes a radiopaque component, and subjecting the target surgical site and the marked location to x-ray exposure to view the target surgical site and mark under x-ray.

### BACKGROUND OF THE INVENTION

Fluoroscopy is a radiological tool used daily in operating rooms. Fluoroscopy is sometimes described as a “live” x-ray. Orthopedic surgeons use fluoroscopy when setting fractures or placing implants; general surgeons use it for insertion of in-dwelling catheters; urologists use it for placement of stents; and cardiologists use it for heart catheterization. Fluoroscopy is ubiquitous in the operating room.

A strong driving force in the evolution of orthopedic procedures has been the push toward minimally invasive surgery. Fluoroscopy has been essential to this push: what you can't see through the incision you can often see with the fluoroscope. Minimally invasive, or percutaneous, orthopedic procedures rely heavily on fluoroscopy.

In order to successfully perform a percutaneous procedure, the incisions must be small and accurate. One of the main roles of fluoroscopy is in selecting the proper incision site. To adhere to minimally invasive standards, orthopedic implants should be guided into bone through precise and strategic incisions.

There are currently two basic ways a surgeon can locate the proper incision site using fluoroscopy: he can poke through the skin with a needle (or other radiopaque instrument), or he can lay a metal object on the skin and expose an image. In either case, the fluoroscopic image will show where the radiopaque object lies in relation to the bone. Both of these techniques have drawbacks. The use of a needle or other probe is invasive, and may lead to pain, hematoma, or infection. Laying an object on the skin causes unnecessary radiation exposure to the surgeon.

U.S. Pat. No. 4,506,676 to Duska describes an adhesive tape having radiopaque material incorporated therein. The tape may be a thin piece of tape that is completely covered with radiopaque material, or may be broader tape with a row of radiopaque dots, or arrow-like markings impregnated into the tape. The tape is placed on the patient's skin near to, but spaced away from an area to be visualized under X-ray, and is used to convey to a radiologist where a physician wants an X-ray to be performed on a patient. This tape is not suggested for use with fluoroscopic procedures, such as during minimally invasive surgery, and is not intended to be located over the surgical site, as the tape may impede surgical procedures to be performed there.

U.S. Pat. No. 5,052,035 to Krupnick discloses a flexible substrate formed of a porous, translucent or transparent material that has lines of a radiopaque material disposed thereon in a grid pattern. In use, the substrate is applied over a selected portion of the body with adhesive patches, and a radiosensitive film is placed under the selected portion of the body. The resulting radiographic image shows the selected body portion with grid indicator lines crossing over it. While this product may be useful for mapping objects taken by X-ray, it is not practical for the minimally invasive type procedures described above, viewed by fluoroscopy.

U.S. Pat. No. 5,193,106 to DeSenna discloses radiopaque material affixed to an adhesive tape and adhered to a patient's foot for use in identifying, under X-ray, deep structures of small bones of the foot. The radiopaque material is formed as stencils of different shapes and sizes that can be placed on the skin, so that the stencil encloses the area to be X-rayed.

U.S. Pat. Nos. 5,383,233; 6,041,094 and 6,985,558 to Russell disclose the use of markers of rubber O-rings or collars (which may include aluminum, for example, or other intermediate density material) to convey pertinent information on radiographic film.

U.S. Pat. No. 5,394,456 to Livingston discloses the use of two overlapping layers having complementary X-ray absorbing characteristics, to form identifying indicia on X-ray film, as the two overlapping layers of material define a predetermined shape. The first layer has a first X-ray absorbing characteristic, and the second layer has a second X-ray absorbing characteristic complementary to the first X-ray absorbing characteristic. The layers are placed to overlap a portion of an X-ray film to function as indicia on the film after exposure of the film.

U.S. Pat. No. 5,469,847 to Zinereich et al. discloses a disk-like adhesive surface marker comprising a gel sealed by a casing and membrane structure. A liquid imaging agent can be injected into a central well of the marker. To use the marker, a backing is removed from the adhesive surface and the marker is then adhered to the patients skin. A liquid imaging agent can be injected into a central well of the marker.

U.S. Patent Application Publication No. US 2004/0127824 to Falahee discloses a marker in the form of a housing including a reservoir filled with a substance, which when dispensed is x-ray visible. The substance is also preferably visible to the unaided eye, and black or pigmented materials may be added to the substance to increase visibility. The substance may be capable of marking on the human skin an on commercially available surgical film. To make the substance opaque, a metal or heavy metal, such as barium may be used. The substance may be dispensed primarily as a liquid or as a powder, and may be erasable. As a liquid, barium particles are suspended in a suitable flowable vehicle. The use of barium or other radiopaque particles in a device of this type may cause clogging of the dispensing tip during use. Also, barium or other radiopaque particles are difficult to maintain in suspension or mixture to ensure that they are equally dispersed throughout a marking liquid of this type. Furthermore only a small volume of the radiopaque substance remains on the substrate (skin or film) after the ink has dried, resulting in a very faint radiographic image. For dispensing powder, there is no disclosure as to how to adhere such a powder to a skin or film.

U.S. Patent Application Publication Nos. US 2004/0253185 and US 2005/0261639 to Herweck et al. disclose a porous applicator that supports capillary action, such that contact of the applicator against a targeted location results in the at least one medicated agent dispensing from the porous applicator to topically apply the at least one medicated agent to the targeted location in a detectable manner. The medicated agent may be included in a radiopaque or otherwise machine detectable ink, so that the stability or migration of the medicated or therapeutic agent applied to a specific targeted location can be confirmed non-invasively by ultrasound, x-ray, MRI, CAT, PET, and the like.

There remains an outstanding need for simple, reliable and inexpensive devices and methods to facilitate the identification of target sites under fluoroscopy.

### SUMMARY OF THE INVENTION

A marking implement is provided for making a radiopaque marking to facilitate a surgical procedure, wherein the implement includes a main body formed of a composition adapted to be smeared onto a substrate when dragged along the substrate, and wherein the composition includes a radiopaque component.

A method of facilitating a surgical procedure is provided, including: subjecting a target surgical site within a patient to x-ray exposure for visualization of the target surgical site; locating a position on the skin of the patient relative to the target surgical site; and marking the position using a marking implement having a main body formed of a composition adapted to be smeared onto the substrate when dragged along the substrate and which includes a radiopaque component.

A method of facilitating a surgical procedure is provided, including: locating a position on the skin of a patient relative to a target surgical site within the patient; marking the position on the skin using the implement of claim 1; and subjecting the target surgical site and the marked location to x-ray exposure to view the target surgical site and mark under x-ray.

These and other features of the invention will become apparent to those persons skilled in the art upon reading the details of the implements and methods as more fully described below.

### BRIEF DESCRIPTION OF THE DRAWINGS

FIG. 1 illustrates an x-ray of a fractured tibia and fibula with pins having been inserted.

FIG. 2 illustrates an x-ray in which screws were placed through small, percutaneous incisions in the skin of the patient.

FIG. 3 is an illustration of a marking implement in accordance with one embodiment of the present invention.

FIG. 4 shows another embodiment of a marking implement according to the present invention.

### DETAILED DESCRIPTION OF THE INVENTION

Before the present implements and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a site” includes a plurality of such sites and reference to “the implement” includes reference to one or more implements and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The present invention is directed to implements and methods for the application of radiopaque markings on a patient which may be referenced by a surgeon during a surgical procedure to assist in more precisely locating subdermal landmarks in a patient.

Radiopaque markings can be made with the implements disclosed herein on the skin of a patient in locations directly overlying surgical target sites to assist the surgeon in making an opening through the patient's skin in a location that is desired, such as a location that is directly aligned with the surgical target for example. With such marks properly positioned, a surgeon can refer to them at any time throughout the surgical procedure, as they will be visible under fluoroscopy. Thus, for example, a surgeon may apply fluoroscopy prior to making an incision and visually verify that a marking on the skin of the patient is aligned with a target location that a surgeon desires to approach by inserting a surgical instrument through an opening in the patient. In this way, smaller, more precise incisions can be made with assurance that an instrument inserted therethrough can be advance directly toward the surgical location.

For example, one or more marks may be made on the skin of a patient in locations directly overlying bone landmarks, such as tuberosities, joint lines, growth plates or fracture lines, to facilitate creation of an opening that directly overlies the landmark of interest. Marks can be made and used in a similar fashion for any other type of surgical target (e.g., organ, vessel, etc.) that a surgeon can view under X-ray or fluoroscopy (such as with the assistance of a contrast agent, for example) to facilitate the proper location to make one or more openings in a patient.

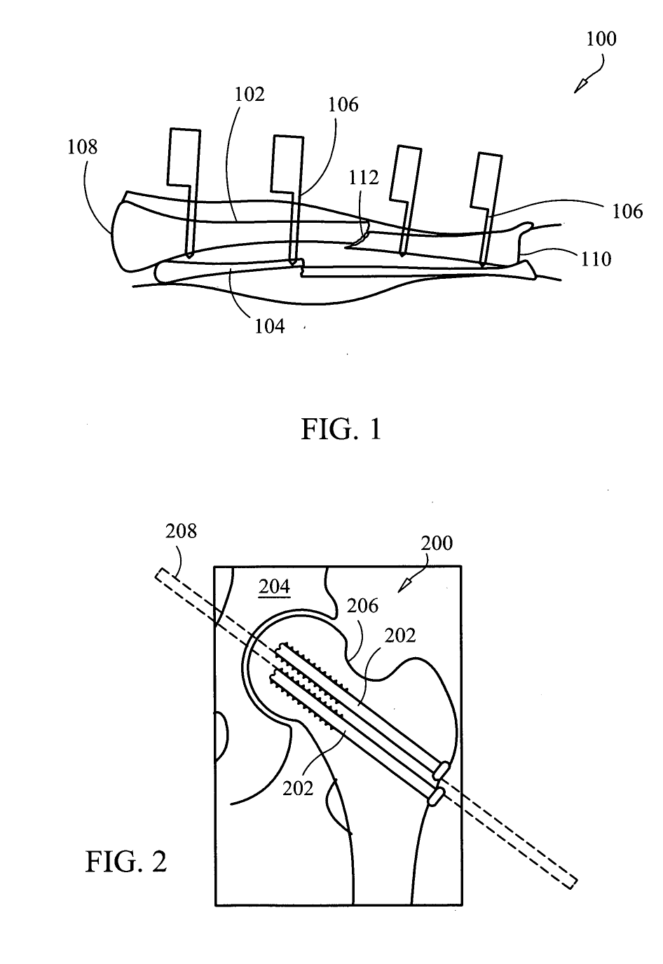

FIG. 1 shows an x-ray 100 of a fractured tibia 102 and fibula 104. Four pins 106 were placed in the tibia 102 only after localizing their proper positions with respect to the knee joint 108, ankle joint 110 and their respective distances from the fracture 112. Localization of the entrance points for insertions of the pins 106 was accompolished by inserting hypodermic needles into the leg under fluoroscopic imaging, and repositioning the needles while visualizing placement, under fluoroscopy, relative to the leg bones, until the needles were determined to be properly positioned. The fluoroscopic imaging was terminated, and ink marks were made on the skin of the leg in the locations where the needles entered when it was determined that they were properly positioned. Such localization can be made much easier by placing the patient's leg under fluoroscopy and then marking the skin of the patient when the implement lines up directly with each of the preferred distances from the fracture 112 that are also aligned over the tibia 102, as all of this can be visualized in real time as the surgeon is holding the marking implement. This also reduces trauma to the patient, by eliminating the need to insert needles by trial and error until the appropriate locations have been determined.

Another important use of a marking implement as described herein is for making directional marks on the skin of the patient. FIG. 2 shows an x-ray 200 in which screws 202 were placed through small, percutaneous incisions in the skin of the patient after a long metal rod 208 (shown in phantom lines) had been positioned by the surgeon directly over the femoral head 204 and neck 206 of the bone, to indicate the angle at which the screws 202 needed to be installed. When this proper angle/direction had been established, a visible ink mark was drawn on the skin and used as a guide during placement of the screws. The present invention can greatly facilitate procedures of this type in that the use of the metal rod 208 can be completely eliminated. Further, the surgeon must expose his hand to radiation using the current practice described, since the metal rod must be held (and maneuvered, if required) until the surgeon determines that the rod has been accurately placed. In contrast, the present invention allows a radiopaque line to be drawn, which allows the surgeon to step back from the radiation field. Thus, not only can radiopaque marks be made on the skin of the patient to indicate where the incision(s) is to be made to align the screws with the bone properly, but a radiopaque directional line can also be drawn on the skin of the patient along the location that the rod 208 would otherwise be placed in, and this can be done in real time, during the procedure, under fluoroscopy.

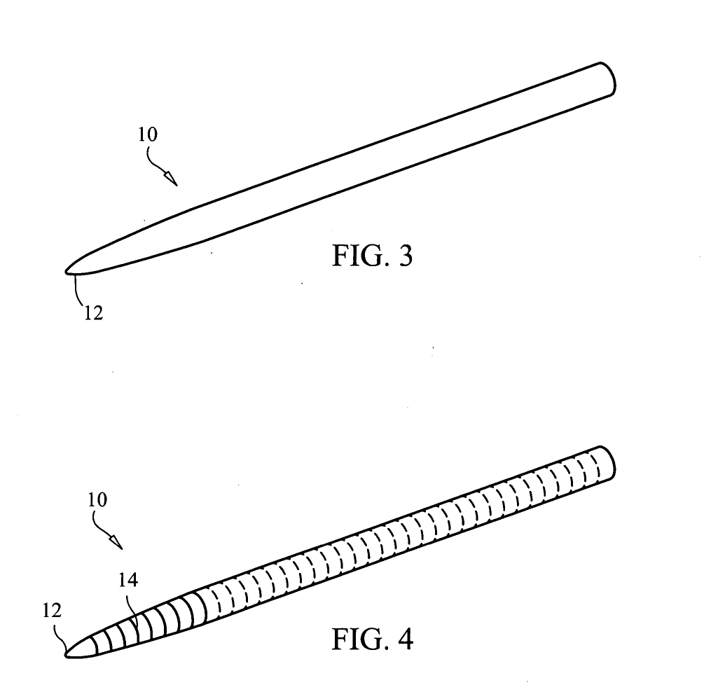

FIG. 3 is an illustration of a marking implement 10 in accordance with one embodiment of the present invention. Implement 10 is elongated, as shown, but need not be, as implement 10 can be formed in any shape as long as a distal tip 12 is provided to extend from the main body of implement 10 that is configured to place marks on the skin of the patient. In the embodiment of FIG. 3 implement 10 is formed as an elongated rod, which is typically substantially cylindrical, but may be oval in cross section, hexagonal in cross-section, or have any other cross-sectional shape desired. Implement 10 is composed of the radiopaque marking composition and does not require a container or reservoir. Distal tip 12 preferably forms a point to enable more precise markings to be made, and this tip may be sharpened, such as by using a knife, scalpel, or other cutting instrument, or even a pencil sharpener having a sufficiently large opening to insert the tip and a portion of the shaft of implement 10 therein.

The marking composition of which implement is made may be a solid or highly viscous liquid that will not deform under its own weight at room temperature. The composition may be a mixture of one or more fillers to provide a relatively greasy or waxy composition, in combination with radiopaque particles (for radiographic identification of the mark) and a colorant (pigment or dye) that can be dispensed upon the skin for visual identification of the mark.

For example, the composition may include one or more of paraffin, beeswax, ceresin, camauba, spermaceti, tallow, potassium carbonate, stearic acid, talc filler, kaolin filler, soap, olive oil, stearic acid, saltpeter and lye, as well as graphite, various inks or dyes for a visual identifier. The radiopaque particles may be made from nearly any metal, preferably biocompatible metals, typically steel or aluminum, although other metals may be used. The metal is formed into a fine particulate or dust that can be readily suspended in the other components when the filler components are melted during the formation stage. The resulting composition is preferably biocompatible and non-toxic.

When cooled to solidify, the composition of implement 10 contains the radiopaque particles and visual colorant dispersed substantially evenly throughout, so that each mark made with the implement on the skin of a patient or radiographic film, for example, applies an even layer of radiographic particles and visual colorant. The consistency of the composition at room temperature should be akin to that of a commercially available grease pencil, or somewhat softer, like a lipstick or other cosmetic.

A mark made by an implement is readily erasable from the skin of a patient or other substrate, such as a plastic film or other substrate by simply wiping away the mark with a dry surgical cloth. This erasability feature is important so that incorrectly placed marks do not radiographically confound the subsequent correct placement of a mark.

FIG. 4 shows another embodiment of implement 10 in which a wrapping 14 is provided around the main body of the implement. Wrapping 14 may be a spirally wrapped paper or plastic cover, or may be form fitted to the main body of the implement, such as a plastic or paper tubing, for example. Wrapping 14 may be either peeled away, or cut away during sharpening and use of the tip 12.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. A marking implement for making a radiopaque marking to facilitate a surgical procedure, said implement comprising:
a main body formed of a composition adapted to be smeared onto a substrate when dragged along the substrate, said composition including a radiopaque component.

2. The marking implement of claim 1, wherein said main body is self-contained so that said composition does not require a container or reservoir.

3. The marking implement of claim 1, wherein said main body is elongated.

4. The marking implement of claim 1, wherein said radiopaque element comprises metallic particles.

5. The marking implement of claim 4, wherein said metallic particles comprise steel or aluminum particles.

6. The marking implement of claim 1, further comprising a wrapping placed around said main body.

7. The marking implement of claim 1, wherein said composition further comprises a component that is visible to the human eye under visible light.

8. The marking implement of claim 1, wherein said composition, after being applied to a substrate, is erasable.

9. The marking implement of claim 1, wherein said composition is erasable with a dry surgical cloth.

10. The marking implement of claim 1, wherein said composition is a solid or highly viscous liquid that is shape retaining at room temperature under its own weight.

11. A method of facilitating a surgical procedure, said method comprising the steps of:
subjecting a target surgical site within a patient to x-ray exposure for visualization of the target surgical site;
locating a position on the skin of the patient relative to the target surgical site; and
marking the position on the skin using the implement of claim 1.

12. The method of claim 11, wherein the location on the skin is aligned with the target surgical site.

13. The method of claim 11, wherein the mark on the position indicates a relative direction or angle along which to perform a surgical step.

14. The method of claim 11, further comprising visualizing the marked position under x-ray to make an incision in the skin.

15. The method of claim 11, further comprising visualizing the marked position under visible light to make an incision in the skin.

16. The method of claim 11, further comprising erasing the mark with a dry cloth to enable remarking without obfuscation by the mark made in claim 11.

17. A method of facilitating a surgical procedure, said method comprising the steps of:
locating a position on the skin of a patient relative to a target surgical site within the patient;
marking the position on the skin using the implement of claim 1; and
subjecting the target surgical site and the marked location to x-ray exposure to view the target surgical site and mark under x-ray.

18. The method of claim 17, wherein the location on the skin is aligned with the target surgical site.

19. The method of claim 17, wherein the mark on the position indicates a relative direction or angle along which to perform a surgical step.
